(19) United States
(12) Patent Application Publication
(10) Pub. No.: US 2022/0108704 A1
(43) Pub. Date: Apr. 7, 2022

(54) REAL-TIME DETECTION AND ALERT OF MENTAL AND PHYSICAL ABUSE AND MALTREATMENT IN THE CAREGIVING ENVIRONMENT THROUGH AUDIO AND THE ENVIRONMENT PARAMETERS

(71) Applicant: Clanz Technology Ltd, Raanana (IL)

(72) Inventors: Nevo Elmalem, Omer (IL); Romi Gubes, Raanana (IL)

(21) Appl. No.: 17/140,165

(22) Filed: Jan. 4, 2021

Related U.S. Application Data
(60) Provisional application No. 63/087,898, filed on Oct. 6, 2020.

(51) Int. Cl. G10L 17/26; G10L 17/06; G10L 21/0208; G10L 15/18; G16H 10/60

(52) U.S. Cl. CPC G10L 17/26; G10L 17/06; G10L 2021/02087; G10L 15/1815; G16H 10/60; G10L 21/0208

(57) ABSTRACT

A computerized-system for detecting low quality of care to a patient and for providing alerts related to the low quality of care, is provided herein. The computerized-system comprising: a recording device; a database of recordings; a database of environment data; a memory to store the plurality of databases; and a processor. The processor is operating a detection and alert module which includes obtaining caregiving environment data from a caregiving environment, by the recording device; collecting environment data manually or from a preconfigured one or more systems. Analyzing each interaction to detect an anomalous behavior in the caregiving environment; and using the collected environment data to classify the detected anomalous behavior, as low quality of care. Upon classification of the detected anomalous data as low quality of care, sending an alert to one or more recipients to be presented on a display unit of a computerized device.

### TECHNICAL FIELD

The present disclosure relates to real-time monitoring systems for real-time detection and alert of mental and physical abuse and maltreatment in the caregiving environment through audio and the environment parameters.

### BACKGROUND

Caregivers help babies, toddlers and adults, such as elderly people and people with disabilities, to carry out activities of daily living. The activities of daily living may include private and intimate tasks, which expose the care recipient to low quality of care given by the caregiver, such as abuse or assault. However, often the care recipients are not in a condition to express or report about the low quality of the care that has been provided.

An ongoing increase in aging population around the world yields a high growth in demand for caregivers. Especially, demand for caregivers having nursing expertise, which may not be replaced by machines and robots. In case of a shortage of professional and qualified workforce, i.e., when demand exceeds supply of appropriate workforce, it may lead to a crisis in the industry of caregivers.

Moreover, the crisis may not only be because demand exceeds supply of appropriate workforce, but also because the need to overcome the shortage of professional and qualified workforce and to fill positions, may be fulfilled by assigning underqualified and nonprofessional caregivers to take care of the people who need help to carry out activities of daily living. This may result in low quality help that may be provided by the underqualified and nonprofessional caregivers which may not be communicated or reported by the care recipients to their responsible adult or guardian.

Currently, there are various solutions for communication and reporting of low quality of care provided by caregivers to a responsible adult or guardian. These solutions, which monitor the caregiving environment, are not reliable, because they are not adjusted to a caregiving environment and may send false-positive alerts to provide immediate intervention when it is actually not needed. For example, a screaming patient having anger bursts may be a normal situation in a caregiving environment and does not require any intervention.

Furthermore, current solutions, which are video based, might have blind spots due to privacy constraints, and may also require constant monitoring. Therefore, these solutions don't detect mental abuse due to privacy violation issues, since they can't be installed in bathrooms and other sensitive private areas.

Accordingly, there is a need for a real-time detection and alert of mental and physical abuse and maltreatment, in a caregiving environment, through audio combined with environment parameters. Furthermore, there is a need that such sound-based detection will be positioned in places where video-based solutions may not be placed due to privacy violation and an alert should be provided to a responsible adult or guardian of the care recipient in case of an event of mental and physical abuse.

Furthermore, for a detection of abnormalities in the caregiving environment, there is a need for automate real-time detection and alert of mental and physical abuse and maltreatment in the caregiving environment through audio combined with the environment parameters, having no human intervention.

Currently, there are systems for monitoring and analyzing audio communication in many fields. For example, in a call center there is an audio analysis to manage agents' performance, U.S. Pat. No. 9,413,891 discloses “ . . . a sentiment to the vocal communication as a function of the acoustical analysis and the presence or absence of specific language, and a display for displaying at least one visual indicator representative of the real time or near real time evaluation of the vocal communication to one of the participants . . . ”.

Another example of audio analysis is the analysis of conversations between customers and call center agents in real-time as disclosed in U.S. Pat. No. 9,160,852. “Expression builder module 170 allows emotion analysis, word/phrase detection, and targeted data detection to be combined . . . reporting of compliancy events by developing a context for detected emotions, words/phrases, and targeted data . . . . By combining real-time emotion, word/phrase, and targeted data analysis of audio data from agents and customers.”

Speech analysis may be also implemented in a speech Neuro-Linguistic Programming (NLP) process to extract sentiment classification from speech as disclosed in U.S. Pat. No. 10,181,333. “a first user at first computer device 130A can be engaged with a speech based teleconference with a second user at computer device 130Z and may be inputting speech based messages into computer device . . . . At block 1021 manager system 110 can activate speech NLP process 111 to extract sentiment classification from speech, e.g. a “fear” sentiment parameter, an “anger” sentiment parameter, a “sadness” sentiment parameter, a “happiness” sentiment parameter, and/or a “disgust” sentiment parameter.”

However, the extraction of sentiment to detect anomalous behavior, based on audio analysis, which is disclosed in these publications is adjusted to call center environment and therefore, the results may not be relevant for a caregiving environment, which is having different parameters for anomalous behavior. Furthermore, none of these publications indicate detection of the level of quality of caregiving interaction in real-time, based on audio analysis combined with environment parameters. Even though the above-mentioned publications use text speech NLP and audio sentiment analysis, none of the publications are adjusted to the caregiving environment by taking into consideration parameters of the environment data. For example, patients in a caregiving environment, may experience outbursts of anger on a daily basis. Current systems which are not adjusted to parameters of the caregiving environment may identify these anger attacks as anomalous behavior and may needlessly send alerts, such as alerts to the patient's guardians or alerts to the employee of the caregiver.

Each caregiving environment has its specific and changing parameters and each one has a different threshold for anomalous behavior. Therefore, there is a need for a technical solution that will aggregate data from different resources and will constantly learn the environment parameters and behavioral patterns to analyze and detect events of anomalous behavior.

### SUMMARY

There is thus provided, in accordance with some embodiments of the present disclosure, a computerized-system for detecting low quality of care and for providing alerts related to the low quality of care.

Furthermore, in accordance with some embodiments of the present disclosure, the computerized-system may include a recording device, a database of recordings, a database of environment data, a memory to store the plurality of databases and a processor. The processor may be configured to operate a detection and alert module.

Furthermore, in accordance with some embodiments of the present disclosure, the detection and alert module may obtain caregiving environment data via a real-time audio stream from the caregiving environment, by the recording device and storing the real-time audio stream in the database of recordings. The environment data may be used to interpret the analyzed data from the real-time audio stream to detect anomalous behavior in the caregiving environment.

Furthermore, in accordance with some embodiments of the present disclosure, the detection and alert module may further collect environment data manually or from a preconfigured one or more systems and may store it in the database of environment data. Then, the detection and alert module may further divide the stored real-time audio stream to one or more segments. Each segment may be related to an interaction which may be between a patient and a caregiver.

Furthermore, in accordance with some embodiments of the present disclosure, the detection and alert module may further analyze each interaction to detect an anomalous behavior, in the caregiving environment and may use the collected environment data to classify the detected anomalous behavior, as low quality of care.

Furthermore, in accordance with some embodiments of the present disclosure, upon classification of the detected anomalous data, as low quality of care, the detection and alert module may send an alert to one or more recipients to be presented on a display unit. The recipients may be the patient's guardians or the employee of the caregiver.

Furthermore, in accordance with some embodiments of the present disclosure, the environment data may be at least one of: patient's personal data; caregiver's personal data; and schedule of patient.

Furthermore, in accordance with some embodiments of the present disclosure, upon classification of the detected anomalous data, as low quality of care, the detection and alert module may maintain the information and send an alert periodically to be presented on a display unit. For example, the alert may be sent to an application that may be running on a user's computerized device, such as, mobile device.

Furthermore, in accordance with some embodiments of the present disclosure, the obtained environment data may be patient's personal data, such as medical condition, caregiver's personal data, such as years of experience, schedule of the care recipient in the care giving environment, etc.

Furthermore, in accordance with some embodiments of the present disclosure, the detection and alert module may further use Artificial Intelligence (AI) models to identify a plurality of speakers in the real-time audio stream and determine a first speaker from the plurality of speakers. The first speaker may be a caregiver or a patient. The AI may be Recurrent Neural Network (RNN) models.

Furthermore, in accordance with some embodiments of the present disclosure, the computerized-system may further comprise a database of voice signatures, and the database of voice signatures may be configured to store voice signatures of all participants in the caregiving environment.

Furthermore, in accordance with some embodiments of the present disclosure, the analyzing of the obtained real-time audio stream may be using machine learning models to compare voice of participants in the interaction in the caregiving environment to pre-collected voice signatures, from the database of voice signatures.

Furthermore, in accordance with some embodiments of the present disclosure, the detection and alert module may be further comprising extracting speech features of the determined first speaker from the real-time audio stream before the analyzing of the obtained real-time audio stream and to identify the one or more participants in an interaction.

Furthermore, in accordance with some embodiments of the present disclosure, the extracted features may be selected from at least one of loudness, pitch, intensity and the like.

Furthermore, in accordance with some embodiments of the present disclosure, the analyzing of the obtained real-time audio stream may be further comprising comparing the extracted features to a preconfigured baseline to yield a sentiment analysis to detect the anomalous behavior.

Furthermore, in accordance with some embodiments of the present disclosure, the anomalous behavior is further detected by a conversational analysis to detect one or more sentiments of the participants in the interaction.

Furthermore, in accordance with some embodiments of the present disclosure, the detection and alert module may further determine one or more additional speech factors. The additional speech factors may be the length of silence between words in the interaction, the number of words in a range of time, loudness, pitch and other features. The other features may be environment features which may be extracted as described in more detail below.

When the result of the comparison may be above a predefined threshold, the detection and alert module may be configured to issue an alert. The alert may be sent to a computerized device of a user, such as a guardian of the care recipient. The alert may be presented on a display unit of a computerized device, such as a mobile device.

According to some embodiments of the present disclosure, the output of the RNN may further provide information concerning abnormalities in the caregiving environment. The information may also include care quality rate, which may be on a range of abuse or maltreatment to positive care, within the caregiving environment. The abnormalities may be anomalous events which comprise one or more identified sentiments.

According to some embodiments of the present disclosure, the caregiving environment may be found among nursing homes, hospitals, private homes, childcare centers and the like.

Furthermore, in accordance with some embodiments of the present disclosure, the computerized-system may further provide, before and after its deployment, said extracted speech features and aggregated contextual environment data to a pre-trained AI models such as Recurrent Neural Network (RNN). The aggregated contextual environment data may be stored in a database.

Furthermore, in accordance with some embodiments of the present disclosure, the RNN may have been previously trained according to a baseline real-time audio stream training data over a predefined period of time, e.g., several days, and the output that the RNN yielded during training, has been compared to expected results.

There is also provided, in accordance with some embodiments of the present disclosure, a computerized-method for detecting low quality of care for a patient and for providing alerts related to the low quality of care.

Furthermore, in accordance with some embodiments of the present disclosure, in a system comprising a recording device; a database of recordings; a database of environment data; a memory to store the plurality of databases; and a processor. The processor may be configured to operate a detection and alert module.

Furthermore, in accordance with some embodiments of the present disclosure, the detection and alert module may be configured to obtain caregiving environment data via a real-time audio stream from the caregiving environment by the recording device. The detection and alert module may further collect environment data manually or from a preconfigured one or more systems and divide the real-time audio stream to one or more segments. Each segment may be related to an interaction.

Furthermore, in accordance with some embodiments of the present disclosure, the detection and alert module may be further configured to analyze each interaction to detect an anomalous behavior, in the caregiving environment and to use the collected environment data to classify the detected anomalous behavior as low quality of care.

Furthermore, in accordance with some embodiments of the present disclosure, upon classification of the detected anomalous data as low quality of care, the detection and alert module may be further configured to send an alert to one or more recipients to be presented on a display unit of a computerized device.

Furthermore, in accordance with some embodiments of the present disclosure, the environment data may be at least one of: patient's personal data; caregiver's personal data; and schedule of patient.

Furthermore, in accordance with some embodiments of the present disclosure, upon classification of the detected anomalous data, as low quality of care, the detection and alert module may maintain the information and may send an alert periodically to be presented on a display unit of a computerized device.

Furthermore, in accordance with some embodiments of the present disclosure, the detection and alert module is using Artificial Intelligence (AI) models to identify a plurality of speakers in the real-time audio stream. The AI models may be Recurrent Neural Networks (RNN).

Furthermore, in accordance with some embodiments of the present disclosure, the detection and alert module is further configured to determine a first speaker from the plurality of speakers.

Furthermore, in accordance with some embodiments of the present disclosure, the system further comprising a database of voice signatures, and wherein said detection and alert module is configured to store voice signatures of all participants in the caregiving environment in the database of voice signatures.

Furthermore, in accordance with some embodiments of the present disclosure, the analyzing of the obtained real-time audio stream may be using machine learning models to compare voice of participants in the interaction in the caregiving environment to pre-collected voice signatures from the database of voice signatures.

Furthermore, in accordance with some embodiments of the present disclosure, the detection and alert module is further configured to extract speech features of the determined first speaker from the real-time audio stream before the analyzing of the obtained real-time audio stream.

Furthermore, in accordance with some embodiments of the present disclosure, the extracted speech features are selected from at least one of: loudness, pitch, intensity and the like.

Furthermore, in accordance with some embodiments of the present disclosure, the analyzing of the obtained real-time audio stream further comprising comparing the extracted features to a preconfigured baseline to yield a sentiment analysis to detect the anomalous behavior.

Furthermore, in accordance with some embodiments of the present disclosure, the anomalous behavior is further detected by a conversational analysis to detect one or more sentiments of the participants in the interaction.

According to some embodiments of the disclosure, the RNN models may be previously trained on a baseline real time audio stream training data over a predefined period of time e.g., several days, and output that has been provided by the RNN models during training has been compared to expected results.

According to some embodiments of the disclosure, the detection and alert model may further compare the received RNN output and the value of the determined additional speech factors. When the comparison is above a predefined threshold, the detection and alert module may issue a real-time alert.

### BRIEF DESCRIPTION OF THE DRAWINGS

In order for the present disclosure, to be better understood and for its practical applications to be appreciated, the following Figures are provided and referenced hereafter. It should be noted that the Figures are given as examples only and in no way limit the scope of the disclosure. Like components are denoted by like reference numerals.

FIG. 1 is a high-level diagram of a computerized-system 100 for detection and alert of mental and physical abuse and maltreatment, in a caregiving environment, through audio combined with environment parameters, in accordance with some embodiments of the disclosure;

FIG. 2 is a high-level illustration of computerized-method for real-time detection and alert of mental and physical abuse and maltreatment, in a caregiving environment, through audio combined with environment parameters, in accordance with some embodiments of the disclosure; and

FIGS. 3A-3B are a flowchart diagram of a computerized method for detecting low quality of care for a patient and for providing alerts related to the low quality of care, in accordance with some embodiments of the disclosure.

### DETAILED DESCRIPTION

In the following detailed description, numerous specific details are set forth in order to provide a thorough understanding of the disclosure. However, it will be understood by those of ordinary skill in the art that the disclosure may be practiced without these specific details. In other instances, well-known methods, procedures, components, modules, units and/or circuits have not been described in detail so as not to obscure the disclosure.

Although embodiments of the disclosure are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the disclosure are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

Some embodiments of the disclosure may include an article such as a computer or processor readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein.

The terms “patient” and “care recipient” are interchangeable.

The terms “AI model” and “machine learning model” are interchangeable.

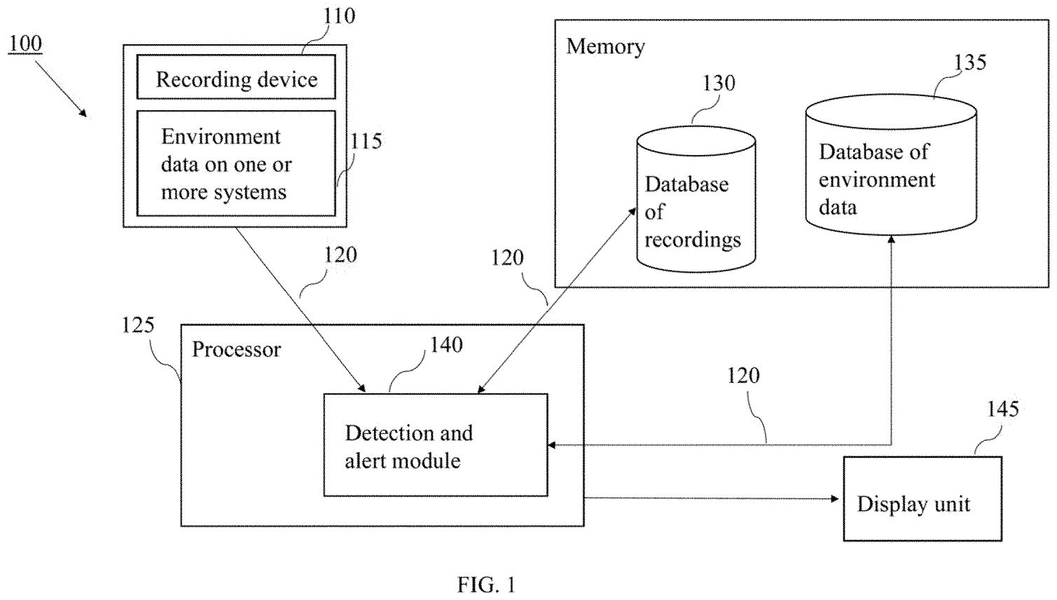

FIG. 1 is a high-level diagram of a computerized-system 100 for real-time detection and alert of mental and physical abuse and maltreatment, in a caregiving environment, through audio combined with environment parameters, in accordance with some embodiments of the disclosure.

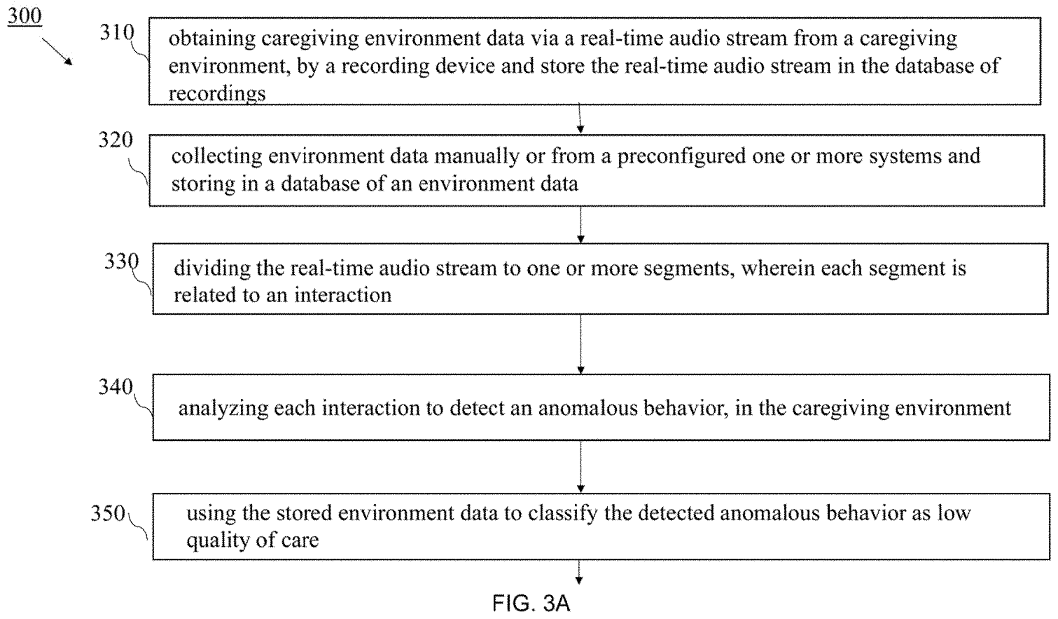

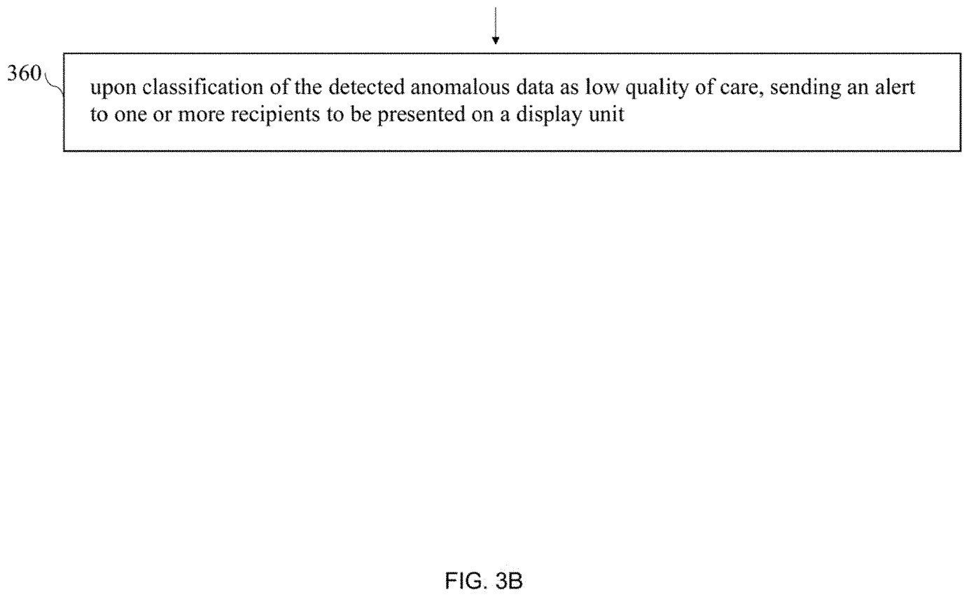

According to some embodiments of the disclosure, in the computerized-system 100 a processor 125 may operate a detection and alert module 140, such as detection and alert module 300 in FIGS. 3A-3B, may collect data, e.g., real-time audio stream, from a caregiving environment via a recording device 110 and manually or from a preconfigured one or more systems 115. The detection and alert module 140 may forward the recordings via electronic communication 120 to a database of recordings, such as database 130, which may be configured to store the real-time audio stream. The detection and alert module 140 may forward the manually collected data and the data from a preconfigured one or more systems via electronic communication 120 to a database, such as database of environment data 135, which may be configured to store the environment data.

According to some embodiments of the disclosure, the detection and alert module 140, such as detection and alert module 300 in FIGS. 3A-3B may perform sound analysis. The sound analysis may include filtering and extracting speech features related to the voices of the caregiver and the patient. For example, loudness, pitch, intensity and the like.

According to some embodiments of the disclosure, the detection and alert module 140, such as detection and alert module 300 in FIGS. 3A-3B may process the extracted speech features along with environment variables as listed below, which may be collected from the caregiver environment by Artificial Intelligence (AI) models.

The AI models may apply machine learning models, deep learning models and other techniques. The AI may be Recurrent Neural Network (RNN).

According to some embodiments of the disclosure, the AI models may provide an identification of each speaker during a caregiving interaction and extract the participant's one or more sentiments within the caregiving environment.

According to some embodiments of the disclosure, the detection and alert module 140, such as detection and alert module 300 in FIGS. 3A-3B, may transcript the interaction, e.g., conversation and extract negative and positive context based on one or more detected sentiments.

According to some embodiments of the disclosure, the detection and alert module 140, such as detection and alert module 300 in FIGS. 3A-3B may perform all the above-mentioned operations for each of the parties or speakers in the caregiving interaction to determine if they have acted in an abusive manner or in a positive manner.

According to some embodiments of the disclosure, the detection and alert module 140, such as detection and alert module 300 in FIGS. 3A-3B, may send the alert upon a determination if a participant has acted in an abusive manner or in a positive manner, as an immediate alert or as a periodical alert to be presented on a computerized device of a user, such as a mobile device. The recipients of the alert, e.g., a user may be the patient's guardians or the employee of the caregiver.

According to some embodiments of the disclosure, the detection and alert module 140, such as detection and alert module 300 in FIGS. 3A-3B, may send the alert to one or more recipients to be presented on a display unit 145 of a computerized device of a user. The computerized device may be a mobile device or any other computerized device.

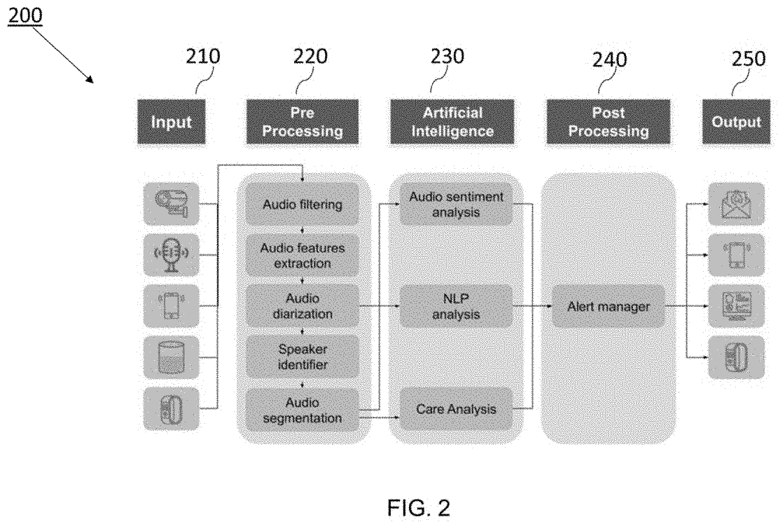

According to some embodiments of the disclosure, the detection and alert module 140, such as detection and alert module 300 in FIGS. 3A-3B, may include an integration with several sources i.e., recording devices, to collect caregiving environment data, such as: Closed Circuit television (CC TV), Internet Protocol (IP) cameras, wearables such as smartwatches, smart home assistance, mobile phones and audio recorders, such as sources 210 in FIG. 2.

FIG. 2 is a high-level illustration of computerized-method 200 for real-time detection and alert of mental and physical abuse and maltreatment, in a caregiving environment, through audio combined with environment parameters, in accordance with some embodiments of the disclosure.

According to some embodiments of the disclosure, the detection and alert module 140, such as detection and alert module 300 in FIGS. 3A-3B, may perform computerized-method 200 to pre-process the audio stream 220 by: (i) performing audio filtering to filter background noises and irrelevant audio segments; (ii) extracting one or more speech features from the audio segments and matching the extracted features to known features; (iii) perform audio diarization; (iv) identify all speakers in the conversation by the comparison of the extracted features to known features; and (v) perform audio segmentation for later on audio sentiment analysis and care analysis by AI model 230.

According to some embodiments of the disclosure, the detection and alert module 140, such as detection and alert module 300 in FIGS. 3A-3B may perform computerized-method 200 to perform audio diarization, which is the process of partitioning an input audio stream into homogeneous segments according to the speaker identity and dividing the audio segments for each speaker. The audio diarization may be performed for later on NLP analysis, by AI module 230.

For each audio segment the following environment features may be extracted:
Frame Energy;
Frame Intensity/Loudness (approximation);
Critical Band spectra (Mel/Bark/Octave, triangular masking filters);
Mel-/Bark-Frequency-Cepstral Coefficients (MFCC);
Auditory Spectra;
Loudness approximated from auditory spectra;
Perceptual Linear Predictive (PLP) Coefficients;
Perceptual Linear Predictive Cepstral Coefficients (PLP-CC);
Linear Predictive Coefficients (LPC);
Line Spectral Pairs (LSP, aka. LSF);
Fundamental Frequency (via ACF/Cepstrum method and via Subharmonic-Summation (SHS));
Probability of Voicing from ACF and SHS spectrum peak;
Voice-Quality: Jitter and Shimmer;
Formant frequencies and bandwidths;
Zero- and Mean-Crossing rate;
Spectral features (arbitrary band energies, roll-off points, centroid, entropy, maxpos, minpos, variance (=spread), skewness, kurtosis, slope);
Psychoacoustic sharpness, spectral harmonicity;
CHROMA (octave warped semitone spectra) and CENS features (energy normalized and smoothed CHROMA);
CHROMA-derived Features for Chord and Key recognition;
F0 Harmonics ratios 1.5. CAPABILITIES—OVERVIEW 9 Video features (low-level);
HSV colour histograms;
Local binary patterns (LBP);
LBP histograms;
Optical flow and optical flow histograms;
Extreme values and positions;
Means (arithmetic, quadratic, geometric);
Moments (standard deviation, variance, kurtosis, skewness);
Percentiles and percentile ranges;
Regression (linear and quadratic approximation, regression error);
Centroid;
Peaks;
Segments;
Sample values;
Times/durations;
Onsets/Offsets;
Discrete Cosine Transformation (DCT);
Zero-Crossings; and
Linear Predictive Coding (LPC) coefficients and gain.

According to some embodiments of the present disclosure, the above-mentioned environment features which may be extracted are features that define a sound signal. These feature are the key for determining a sentiment and events like shower, cry, falling person and the like, by the computerized-system 100 in FIG. 1, which may perform computerized-method 200 for detecting low quality of care and providing real time alerts related to the low quality of care, that is provided in a caregiving environment. For each segment of sound in the interaction, the above-mentioned environment features may be extracted. The AI model may learn for each class what are the environment feature values that characterize it.

According to some embodiments of the present disclosure, the AI model may find the relevant the environment features which were extracted from each audio segment according to one or more training datasets, which have been previously provided to the AI model, during the training phase of the AI model. For example, the one or more training datasets may include audio segments having angry participants in which the participants were tagged as angry, the AI model may identify the relevant features which are related to anger in speech. When the AI may be operated to analyze an interaction between participants in a caregiving environment, the AI may detect an anomalous behavior. For example, the AI model may identify a sentiment such as anger among the participants in the audio segments, and mat detect an anomalous behavior.

According to some embodiments of the present disclosure, in some instances, computerized-method 200 for detecting low quality of care may not send an alert related to the low quality of care. The computerized-method 200 for detecting low quality of care may not send an alert based on the detected anomalous behavior only, but also take into consideration the environment data which has been collected manually or from a preconfigured one or more systems and stored in the database of the environment data. For example, in case a sentiment such as anger has been leading to a detection of anomalous behavior, but the environment data may indicate that one of the participants is suffering from Tourette syndrome and hence involve unwanted sounds that can't be controlled the computerized-method 200 for detecting low quality of care will not send an alert of low-quality of care to one or more recipients to be presented on a display unit.

According to some embodiments of the present disclosure, the determined sentiment along with the events and the environmental data are used by the computerized-system 100 in FIG. 1 and computerized-method 200 for detecting low quality of care and providing real time alerts related to the low quality of care, that is provided in a caregiving environment.

According to some embodiments of the present disclosure, the detection and alert module 140 may perform computerized-method 200 for detecting low quality of care and providing real time alerts related to the low quality of care, that is provided in a caregiving environment, may implement AI module 230, which may apply machine learning models to detect low quality of care. The machine learning models may determine one or more sentiments for each participant during an interaction in a caregiving environment, based on the extracted environment features, mentioned above.

According to some embodiments of the present disclosure, each of the one or more sentiments may be determined, for each participant, by a combination of different extracted features having different values. Meaning, one combination of extracted features may be interpreted by the AI module 230, as a certain sentiment for one participant but may not be interpreted as that sentiment to one or more of the other participants.

For each participant the detection and alert module 140 in FIG. 1, may compare a list of features which were set for each participant's talk. Thus, for each participant there may be different values which may have been previously set for the list of features. For example, a feature, such as frame energy may be extracted for two participants of an interaction. The value of the frame energy may have been set for one participant to define the first participant's talk as high in the aspect of the energy feature and the value of the frame energy may have been set for one participant to define the second participant's talk as low in the aspect of the energy feature, because the second participant speaks quieter than the first participant. For each participant there may be different values in the list of features, which have been previously set.

According to some embodiments of the disclosure, the machine learning models may detect an event where low quality of care have been provided by a different combination of sentiment of each participant in the event or interaction.

According to some embodiments of the disclosure, the detection and alert module 140 in FIG. 1, may perform computerized-method 200 to operate unsupervised AI models to cluster each word in the recorded caregiving interaction to a certain speaker.

According to some embodiments of the disclosure, the detection and alert module 140 in FIG. 1, may perform computerized-method 200 to identify each speaker by comparing extracted features from the recorded voice during the caregiving interaction and compare it to pre-collected voice signatures stored in a data storage (not shown).

According to some embodiments of the disclosure, the detection and alert module 140 in FIG. 1, may perform computerized-method 200 to receive parameters from the employer of the caregiver that may be inserted manually or received from integrated systems and store it in a data storage, such as database 130 in FIG. 1. The parameters may include personal information of the caregivers and patients, schedule and information of location of the caregiving environment and staff.

According to some embodiments of the disclosure, the detection and alert module 140 in FIG. 1, may perform computerized-method 200 to provide the extracted speech features and contextual environment data to a pretrained RNN such as AI module 230 in FIG. 2 to identify anomalous behavior.

According to some embodiments of the disclosure, the RNN may be trained according to a baseline real-time audio stream training data, over a predefined period of time, such as, several days and may be forwarded as an output to the detection and alert module 140 in FIG. 1, which may perform computerized-method 200. During the training tagged datasets may assist the machine learning model to identify which features are related to each sentiment and to detect anomalous behavior in an analyzed interaction.

According to some embodiments of the disclosure, the output of the RNN may be considered as a score to represent a margin from the baseline. Another score may be provided from the textual context when NLP analyzes the text received from transcripting the speaker's segments by modules such as Artificial Intelligence (AI) module 230.

According to some embodiments of the disclosure, the detection and alert module 140 in FIG. 1, may perform computerized-method 200 to receive information from the AI module 230 and accordingly determine whether or not to issue and display a real-time alert according to a predefined threshold such as alert manager module which is a post processing module 240.

According to some embodiments of the disclosure, a real-time alert may be applied with common communication methods such as a phone call, a Short Message Service (SMS), email, WhatsApp application, Telegram application and Facebook, for example via a mobile phone 140 in FIG. 1 and output 250. The alert may also be presented via other devices such as computer display screen, smartwatch and the like.

According to some embodiments of the disclosure, the implementation of the detection and alert module 140 in FIG. 1, may perform computerized-method 200 which may be operated in a system for monitoring the quality of care for elders, children and people with disabilities, both in private homes and care institutions, such as nursing homes, long term care facilities, daycare centers, hospitals, hospices, childcare centers.

The implementation of computerized-method 200 may be used for example, by the elder's children who live far away and are worried about their parents. The children might want to make sure that their parents are well treated.

FIGS. 3A-3B are a flowchart diagram of a computerized method for detecting low quality of care for a patient and for providing alerts related to the low quality of care, in accordance with some embodiments of the disclosure.

According to some embodiments of the disclosure, operation 310 may comprise obtaining caregiving environment data via a real-time audio stream from a caregiving environment, by a recording device and store the real-time audio stream in the database of recordings.

According to some embodiments of the disclosure, operation 320 may comprise collecting environment data manually or from a preconfigured one or more systems and storing in a database of an environment data.

According to some embodiments of the disclosure, operation 330 may comprise dividing the real-time audio stream to one or more segments, wherein each segment is related to an interaction.

According to some embodiments of the disclosure, operation 340 may comprise analyzing each interaction to detect an anomalous behavior, in the caregiving environment.

According to some embodiments of the disclosure, operation 350 may comprise using the stored environment data to classify the detected anomalous behavior as low quality of care.

According to some embodiments of the disclosure, operation 360 may comprise upon classification of the detected anomalous data as low quality of care, sending an alert to one or more recipients to be presented on a display unit.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Different embodiments are disclosed herein. Features of certain embodiments may be combined with features of other embodiments; thus certain embodiments may be combinations of features of multiple embodiments. The foregoing description of the embodiments of the disclosure has been presented for the purposes of illustration and description. It is not intended to be exhaustive or to limit the disclosure to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the disclosure. While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

What is claimed:

1. A computerized-system for detecting low quality of care to a patient and for providing alerts related to the low quality of care, the computerized-system comprising:
a recording device;
a database of recordings;
a database of environment data;
a memory to store the plurality of databases; and a processor, said processor is configured to operate a detection and alert module, said detection and alert module comprising:
obtaining caregiving environment data via a real-time audio stream from a caregiving environment, by the recording device and storing the real-time audio stream in the database of recordings;
collecting environment data manually or from a preconfigured one or more systems and storing in the database of the environment data;
dividing the stored real-time audio stream to one or more segments, wherein each segment is related to an interaction;
analyzing each interaction to detect an anomalous behavior, in the caregiving environment; and
using the stored environment data to classify the detected anomalous behavior as low quality of care,
wherein upon classification of the detected anomalous data as low quality of care, sending an alert to one or more recipients to be presented on a display unit.

2. The computerized-system of claim 1, wherein the environment data is at least one of: patient's personal data; caregiver's personal data; and schedule of patient.

3. The computerized-system of claim 1, wherein upon classification of the detected anomalous data, as low quality of care, maintaining the information and sending an alert periodically to be presented on a display unit.

4. The computerized-system of claim 1, wherein the detection and alert module is using Artificial Intelligence (AI) models to identify a plurality of speakers in the real-time audio stream.

5. The computerized-system of claim 4, wherein the detection and alert module is further configured to determine a first speaker from the plurality of speakers.

6. The computerized-system of claim 1, wherein the computerized-system further comprising a database of voice signatures, and wherein said database of voice signatures is configured to store voice signatures of all participants in the caregiving environment.

7. The computerized-system of claim 6, wherein the analyzing of the interaction is using machine learning models to compare voice of participants in the interaction in the caregiving environment to pre-collected voice signatures from the database of voice signatures.

8. The computerized-system of claim 5, wherein the detection and alert module is further comprising extracting speech features of the determined first speaker from the real-time audio stream before the analyzing of the interaction.

9. The computerized-system of claim 8, wherein the extracted speech features are selected from at least one of: loudness, pitch, intensity and the like.

10. The computerized-system of claim 8, wherein the analyzing of interaction further comprising comparing the extracted features to a preconfigured baseline to yield a sentiment analysis to detect the anomalous behavior.

11. The computerized-system of claim 10, wherein the analyzing each interaction to detect an anomalous behavior further comprises a conversational analysis to detect one or more sentiments of the participants in the interaction.

12. A computerized-method for detecting low quality of care for a patient and for providing alerts related to the low quality of care, the computerized-method comprising:
in a system comprising a recording device; a database of recordings; a database of environment data; a memory to store the plurality of databases; and a processor, said processor is configured to operate a detection and alert module, said detection and alert module is configured to:
obtain caregiving environment data via a real-time audio stream from a caregiving environment, by the recording device and store the real-time audio stream in the database of recordings;
collect environment data manually or from a preconfigured one or more systems and storing in the database of the environment data;
divide the stored real-time audio stream to one or more segments, wherein each segment is related to an interaction;
analyze each interaction to detect an anomalous behavior, in the caregiving environment; and
use the stored environment data to classify the detected anomalous behavior as low quality of care,
wherein upon classification of the detected anomalous data as low quality of care, send an alert to one or more recipients to be presented on a display unit.

13. The computerized-method of claim 12, wherein the environment data is at least one of: patient's personal data; caregiver's personal data; and schedule of patient.

14. The computerized-method of claim 12, wherein upon classification of the detected anomalous data, as low quality of care, maintaining the information and sending an alert periodically to be presented on a display unit.

15. The computerized-method of claim 12, wherein the detection and alert module is using Artificial Intelligence (AI) models to identify a plurality of speakers in the real-time audio stream.

16. The computerized-method of claim 15, wherein the detection and alert module is further configured to determine a first speaker from the plurality of speakers.

17. The computerized-method of claim 16, wherein the analyzing of the interaction is using machine learning models to compare voice of participants in the interaction in the caregiving environment to pre-collected voice signatures from the database of voice signatures.

18. The computerized-method of claim 15, wherein the detection and alert module is further comprising extracting speech features of the determined first speaker from the real-time audio stream before the analyzing of the interaction.

19. The computerized-method of claim 18, wherein the extracted speech features are selected from at least one of: loudness, pitch, intensity and the like.

20. The computerized-system of claim 18, wherein the analyzing of interaction further comprising comparing the extracted features to a preconfigured baseline to yield a sentiment analysis to detect the anomalous behavior.

21. The computerized-system of claim 20, wherein the analyzing each interaction to detect an anomalous behavior further comprises a conversational analysis to detect one or more sentiments of the participants in the interaction.
